(19) United States
(12) Patent Application Publication
(10) Pub. No.: US 2026/0358004 A1
(43) Pub. Date: Nov. 12, 2026

(54) AIR CONDITIONER AND OPERATING METHOD THEREFOR

(71) Applicant: LG ELECTRONICS INC., Seoul (KR)

(72) Inventors: Ilna SON, Seoul (KR); Chulwoo PARK, Seoul (KR); Yumin KIM, Seoul (KR)

(21) Appl. No.: 19/109,204

(22) PCT Filed: Sep. 7, 2023
(86) PCT No.: PCT/KR2023/013385

§ 371 Date: Mar. 6, 2025

(30) Foreign Application Priority Data
Sep. 8, 2022 (KR) 10-2022-0113994

(51) Int. Cl. F24F 6/12; A61L 2/10; A61L 103/00; F24F 6/00; G01N 21/47; G01N 33/18

(52) U.S. Cl. CPC F24F 6/12; A61L 2/10; G01N 21/4738; G01N 33/18; A61L 2103/23; A61L 2202/11; F24F 2006/006

(57) ABSTRACT

An air conditioner according to an embodiment of the present disclosure includes: a water tank in which water that is generated or required in the process of conditioning the air is accommodated; a turbidity sensor arranged under the lower end portion of the water tank; a sensor water-supply flow path through which the water accommodated in the water tank flows to the turbidity sensor; a valve opening/closing the sensor water-supply flow path; a sensor drainage flow path through which the water discharged from the turbidity sensor flows; and a pump allowing the water discharged from the turbidity sensor to flow to the water tank through the sensor drainage flow path.

### TECHNICAL FIELD

The present disclosure relates to an air conditioner and an operating method therefor, and more particularly, to an air conditioner including a water tank and a sensor capable of discriminating a contamination level of water in the water tank and an operating method therefor.

### BACKGROUND ART

An air conditioner is a device that changes indoors into a comfortable environment by flowing the air and performing at least one of cooling, heating, air purification, humidification, or dehumidification. The air conditioner has a water tank that stores water for humidification or dehumidification. Since the water tank always contains water and is in contact with the air, it is easy for microorganisms to propagate. As a result, a user has to clean the water tank unnecessarily frequently, which is inconvenient for maintenance. Accordingly, various studies are being conducted for water tank management.

The related art of Korean Patent No. 10-1073637 discloses an air conditioner comprising both a large humidifier and a small humidifier as a humidifying means. A beam sensor is applied to manage a humidifier water tank. However, instead of determining the contamination level of the water inside the water tank, the Korean patent merely determines the contamination of an inner wall surface of the water tank.

The related art of Korean Patent Application Publication No. 10-2018-0134186 discloses a pollution measuring device measuring a pollution level of water accommodated in a humidifier water tank. However, the pollution measuring device installed on the surface of the water tank may experience measurement errors when there is internal flow, and its measured value may be greatly affected by the amount of water in the water tank.

### DISCLOSURE

### Technical Problem

An aspect of the present disclosure is directed to providing an air conditioner capable of accurately discriminating a contamination level of water in a water tank.

Yet another aspect of the present disclosure is directed to providing an air conditioner capable of easily managing a water tank.

Yet another aspect of the present disclosure is directed to providing an air conditioner capable of enhancing internal hygiene performance and resolving hygiene-related inconveniences in use.

Yet another aspect of the present disclosure is directed to providing an air conditioner capable of determining a contamination level of water in a water tank and presenting management feedback.

The aspects of the present disclosure are not limited to those mentioned above, and other aspects not mentioned may be clearly understood by those skilled in the art from the following description.

### Technical Solution

An air conditioner according to embodiments of the present disclosure may improve hygiene and usability by applying a sensor that senses the state of water in a water tank.

The air conditioner according to the embodiments of the present disclosure configures a separate flow path for sampling the water in the water tank, thereby minimizing the influence of sensing according to the amount of water and accurately discriminating a contamination level of water.

The air conditioner according to the embodiments of the present disclosure may minimize sensing errors with an operating logic suitable for sensing the contamination level of water.

The air conditioner according to the embodiments of the present disclosure includes: a water tank accommodating water; a turbidity sensor arranged under the lower end portion of the water tank; a sensor water-supply flow path through which the water accommodated in the water tank flows to the turbidity sensor; and a valve opening/closing the sensor water-supply flow path.

The air conditioner according to the embodiments of the present disclosure includes: a water tank in which water that is generated or required in the process of conditioning the air is accommodated; a turbidity sensor arranged under the lower end portion of the water tank; a sensor water-supply flow path through which the water accommodated in the water tank flows to the turbidity sensor; a valve opening/closing the sensor water-supply flow path; a sensor drainage flow path through which the water discharged from the turbidity sensor flows; and a pump allowing the water discharged from the turbidity sensor to flow to the water tank through the sensor drainage flow path.

The turbidity sensor may include a chamber filled with water supplied from the water tank, a light source unit including a light source that irradiates light to an interior of the chamber, and a light receiving unit that receives scattered light scattered in the chamber.

The light source unit may irradiate the light when the chamber is filled to a certain water level or higher.

The turbidity sensor may sense and output turbidity of water supplied from the water tank after a preset stabilization time has elapsed.

The valve may open the sensor water-supply flow path and close the sensor water-supply flow path after a preset water supply standard time has elapsed.

The air conditioner according to an embodiment of the present disclosure may further include a controller that discriminates a microbial contamination level in the water tank based on a sensing value of the turbidity sensor and controls to perform a feedback operation in response to the discriminated microbial contamination level.

The controller may turn off an operation of the air conditioner and control an opening of the valve.

The air conditioner according to an embodiment of the present disclosure may further include a UV module that irradiates ultraviolet rays to an interior of the water tank when the discriminated contamination level is included in a first section.

The air conditioner according to an embodiment of the present disclosure may further include an output module that displays washing-related information corresponding to the discriminated contamination level when the discriminated contamination level is included in a second section higher than the first section.

The air conditioner according to an embodiment of the present disclosure may further include a communication module that automatically requests a care service in response to the discriminated contamination level when the discriminated contamination level is included in a third section higher than the second section.

A method for operating an air conditioner according to an embodiment of the present disclosure includes: operating a valve to open a sensor water-supply flow path connecting a water tank in which water that is generated or required in the process of conditioning the air is accommodated and a turbidity sensor arranged under the lower end portion of the water tank; operating the valve to close the sensor water-supply flow path; using the turbidity sensor to sense turbidity of water supplied from the water tank when a stabilization time has elapsed; and performing a feedback operation based on a sensing value of the turbidity sensor.

The method for operating an air conditioner according to an embodiment of the present disclosure may further include draining of discharging water from the turbidity sensor.

The method for operating an air conditioner according to an embodiment of the present disclosure may further include turning off an operation of the air conditioner before opening the sensor water-supply flow path.

The operation of the valve to close the sensor water-supply flow path may change an operating state of the valve when a preset water supply standard time has elapsed after opening or when an internal chamber of the turbidity sensor is filled with water exceeding a certain amount.

The use of the turbidity sensor to sense the turbidity of the water supplied from the water tank may include: irradiating light to an interior of the chamber; receiving scattered light scattered in the chamber; and analyzing a pattern of the received scattered light.

The performance of the feedback operation may include displaying a discriminated contamination level and irradiating ultraviolet rays to an interior of the water tank when the discriminated contamination level is included in a first section.

The performance of the feedback operation may include displaying discriminated contamination level information and information guiding washing of the water tank when the discriminated contamination level is included in a second section higher than the first section.

The performance of the feedback operation may include transmitting a signal to a server to transmit a washing guide image of the water tank to a preset device.

The performance of the feedback operation may include displaying the discriminated contamination level information and automatically requesting a care service in response to the discriminated contamination level when the discriminated contamination level is included in a third section higher than the second section.

### Advantageous Effects

According to at least one of the embodiments of the present disclosure, a contamination level of water in a water tank can be accurately discriminated.

In addition, according to at least one of the embodiments of the present disclosure, the water tank can be easily managed.

In addition, according to at least one of the embodiments of the present disclosure, internal hygiene performance can be enhanced and hygiene-related inconveniences in use can be resolved.

In addition, according to at least one of the embodiments of the present disclosure, the contamination level of water in the water tank can be determined and management feedback can be presented.

Various other benefits will be described directly or implicitly in the detailed description according to the embodiments of the present disclosure to be described later.

### DESCRIPTION OF DRAWINGS

FIG. 1 is a conceptual diagram illustrating an air conditioner according to an embodiment of the present disclosure.

FIG. 2 is a flowchart of an operating method of an air conditioner according to an embodiment of the present disclosure.

FIG. 3 is a perspective view of an air conditioner according to an embodiment of the present disclosure.

FIG. 4 is an exploded perspective view of the air conditioner of FIG. 3.

FIG. 5 is a conceptual diagram of an air conditioner according to another embodiment of the present disclosure.

FIG. 6 is a conceptual diagram of a turbidity sensor according to an embodiment of the present disclosure.

FIG. 7 is a flowchart of an operating method of an air conditioner according to an embodiment of the present disclosure.

FIGS. 8 to 11 are diagrams for reference in describing the operation of an air conditioner according to an embodiment of the present disclosure.

### MODE FOR DISCLOSURE

Hereinafter, the exemplary embodiments of the present disclosure are described in detail with reference to the accompanying drawings. The present disclosure may, however, be embodied in many different forms and should not be construed as being limited to the embodiments set forth herein.

In order to clearly and briefly describe the present disclosure, the parts irrelevant to the description are omitted in the drawings. The same reference numerals are used to designate the same or similar parts throughout the specification.

With respect to constituents used in the following description, the suffixes “module” and “unit” are merely given in consideration of only facilitation of description and do not have any special importance or role. Accordingly, the “module” and the “unit” may be used interchangeably.

In addition, it will be understood that, although the terms “first”, “second”, etc. may be used herein to describe various elements, these elements should not be limited by these terms. These terms are only used to distinguish one element from another element.

In addition, the term “air conditioner” used herein refers to a device having a humidifying function or a dehumidifying function, and may include a humidifier, a dehumidifier, an air purifier and an chiller/heater having a dehumidifying and/or humidifying function.

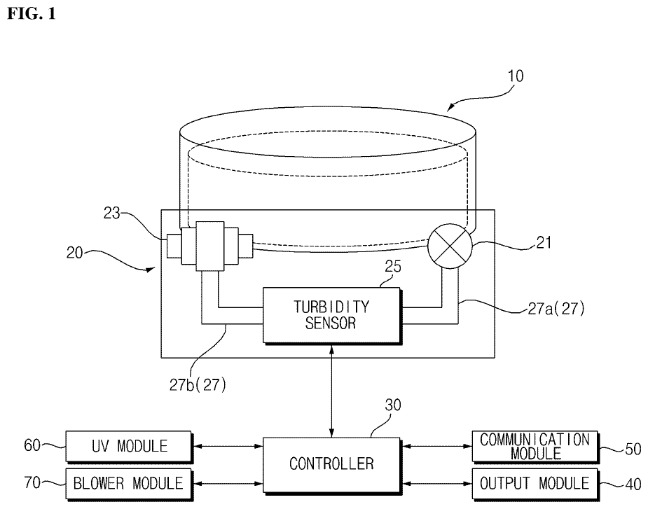

FIG. 1 is a conceptual diagram illustrating an air conditioner according to an embodiment of the present disclosure.

Referring to FIG. 1, an air conditioner 1 according to embodiments of the present disclosure includes a water tank 10 accommodating water. In the water tank 10, water that is generated or required in the process of conditioning the air is accommodated. For example, the water needed to increase the humidity of the air may be stored in the water tank 10. Alternatively, the water generated during a dehumidification process may be accommodated in the water tank 10.

In addition, the air conditioner 1 includes a water tank sensing module 20 that discriminates a contamination level of water in the water tank 1. In order to accurately discriminate the contamination level of water in the water tank 1, the water tank sensing module 20 includes a turbidity sensor 25 and a sensor flow path 27.

The air conditioner 1 includes the turbidity sensor 25 arranged under the lower end portion of the water tank 10, and the water tank 10 and the turbidity sensor 25 are connected through the sensor flow path 27.

The sensor flow path 27 includes a sensor water-supply flow path 27a through which the water accommodated in the water tank 10 flows to the turbidity sensor 25, and the sensor water-supply flow path 27a includes an opening/closing valve 21 opening/closing the sensor water-supply flow path 27a. The opening/closing valve 21 may be controlled to open to supply water to the turbidity sensor 25 for a predetermined time. The opening/closing valve 21 may open the sensor water-supply flow path 27a and close the sensor water supply path 27a after a preset water supply standard time has elapsed. Accordingly, a certain amount of water may be supplied to the turbidity sensor 25. The water supply standard time may be decided in advance based on the amount of water needed for sensing by the turbidity sensor 25 and the speed at which water enters an interior of the turbidity sensor 25.

Since the turbidity sensor 25 is arranged under the lower end portion of the water tank 10, when the opening/closing valve 21 is controlled to open and the sensor water-supply flow path 27a is opened, water inside the water tank 10 may be supplied to the turbidity sensor 25 along the sensor water-supply flow path 27a without any other power. The turbidity sensor 25 may sense the turbidity of water entered through the sensor water-supply flow path 27a.

In addition, since the turbidity sensor 25 is arranged under the lower end portion of the water tank 10 and a certain amount of water may be supplied through the sensor water-supply flow path 27a, the contamination level of water may be discriminated regardless of the amount of water accommodated in the water tank 10. In other words, by arranging a separate flow path 27a for sampling water in the water tank and the turbidity sensor 25 at a lower side of the water tank 10, the influence of sensing according to the amount of water is minimized, thereby accurately discriminating the contamination level of water.

The sensor flow path 27 further includes a sensor drainage flow path 27b through which the water discharged from the turbidity sensor 25 flows. The air conditioner 1 according to an embodiment of the present disclosure further includes a pump 23 allowing the water discharged from the turbidity sensor 25 to flow to the water tank 20 through the sensor drainage flow path 27b, with the sensor drainage flow path 27b being connected to the water tank 10. The pump 23 includes a motor and may draw water from a lower side to an upper side. The pump 23 may be operated so that water is circulated back from the turbidity sensor 25 to the water tank 10. The pump 23 may be arranged inside the water tank sensing module 20 or inside the water tank 10 and connected to the sensor drainage flow path 27b. According to an embodiment, the sensor drainage flow path 27b may also discharge water discharged from the turbidity sensor 25 to the outside.

Referring to FIG. 1, the air conditioner 1 may further include a controller 30, an output module 40, a communication module 50, a blower module 60, and a UV module 60.

The output module 40 may be provided with a display device such as a display (not shown) or a light emitting diode (LED) (not shown). For example, the output module 40 may display information such as the driving state of the air conditioner 1, the operating state related to the occurrence of an error, or the contamination level of water.

The output module 40 may be provided with an audio device such as a speaker (not shown) or a buzzer (not shown). For example, the output module 40 may output a sound effect regarding the driving state of the air conditioner 1 and may output a predetermined warning sound when an error occurs.

The communication module 50 may provide an interface for communicating with other devices. For example, the communication module 50 may provide an interface for transmitting and receiving signals according to the RS-485 communication method, which is a serial communication protocol standard that supports a home network. For example, the communication module 50 may transmit and receive signals with at least one of the other devices using a wireless communication method such as Wi-Fi, Bluetooth, beacon, or Zigbee.

The UV module 60 eliminates bacteria or viruses by irradiating ultraviolet rays. The UV module 60 may irradiate ultraviolet rays toward water stored in the water tank 10 to eliminate bacteria or viruses that propagate in the water accommodated in the water tank 10.

The blower module 70 is intended to form an air flow to allow external air to enter the air conditioner 1, and may include a blower fan (not shown) that blows the air and a blower motor (not shown) that rotates the blower fan.

The controller 30 may be connected to each component provided in the air conditioner 1. For example, the controller 30 may transmit and/or receive signals between each component provided in the air conditioner 1 and control the overall operation of each component.

The controller 30 may include at least one processor, and may control the overall operation of the air conditioner 1 using the processor included therein. Herein, the processor may be a general processor such as a central processing unit (CPU). The processor may be a dedicated device such as an ASIC, or may be any of other hardware-based processors.

The controller 30 may perform various calculations based on data received through a sensor unit (not shown) including various sensors such as the turbidity sensor 25 and the communication module 50. In addition, the controller 30 may store data received through the sensor unit (not shown) such as the turbidity sensor 25 and the communication module 50 in a memory (not shown).

There is the turbidity sensor 25 at a lower portion of the water tank 10 to sense the state of the water in the water tank 10 and determine the state of the water, such as the level of microorganisms and whether there is an odor. The turbidity sensor 25 may sense the turbidity of water entered through the sensor water-supply flow path 27a and output the same to the controller 30)

The controller 30 may discriminate a microbial contamination level in the water tank based on a sensing value of the turbidity sensor 25 and control to perform a feedback operation in response to the discriminated microbial contamination level. Alternatively, the turbidity sensor 25 may directly discriminate the microbial contamination level and transmit the same to the controller 30, and the controller 30 may control other components to perform appropriate feedback operations based on the received microbial contamination level.

The controller 30 may control the output module 50 and/or the communication module 50 to recognize the contamination state of water accommodated in the water tank 10 and provide a user with information on a cleaning alarm or water replacement cycle.

In addition, the controller 30 may sense in advance any odor that may occur in the water tank 10 depending on the contamination level water accommodated in the water tank 10, and operate the automatic washing/sterilization logic through the UV module 60 before a customer feels so, or request a care service from a professional through the communication module 50. Accordingly, the convenience and hygiene of use for non-professional users may be improved.

For example, the contamination level may be divided into three sections as shown below, and the controller 30 may be controlled to perform a feedback operation according to the section corresponding to the sensing result.
High contamination level (third zone): Automatically call a care service
Medium contamination level (second section): Alarm and provide information for user washing progress
Low contamination level (first section): Automatic washing logic

The controller 30 may control the output module 40 so that the output module 40 displays information corresponding to the discriminated contamination level.

When the discriminated contamination level is included in the first section, which is the section with the lowest contamination level, the controller 30 may control the UV module 60 to irradiate ultraviolet rays to an interior of the water tank 10. The UV module 60 may adjust the output of ultraviolet rays in response to the discriminated contamination level. Accordingly, more efficient sterilization/washing is possible.

In addition, when the discriminated contamination level is included in the second section that is higher than the first section, the controller 30 may control the output module 40 so that the output module 40 displays washing-related information corresponding to the discriminated contamination level. Here, the washing-related information may include at least one of a washing-required situation alarm, a washing-related video, or washing-related link information.

When the discriminated contamination level is included in the third section that is higher than the second section, the controller 30 may control the communication module 50 to automatically request a care service in response to the discriminated contamination level.

The controller 30 may turn off the humidification or dehumidification operation of the air conditioner 1 and control to open the opening/closing valve 21 to improve the accuracy of water contamination level discrimination.

In addition, the turbidity sensor 25 may sense and output the turbidity of water supplied from the water tank 10 after a preset stabilization time has elapsed. Immediately after water supply, bubbles and vortices may occur due to the flow of water, which may reduce the accuracy of water quality sensing. Accordingly, it is preferable that the turbidity sensor 25 performs sensing after water supply is completed and a predetermined time has elapsed. The stabilization time may be decided in advance based on the amount and speed of water entering an interior of the turbidity sensor 25.

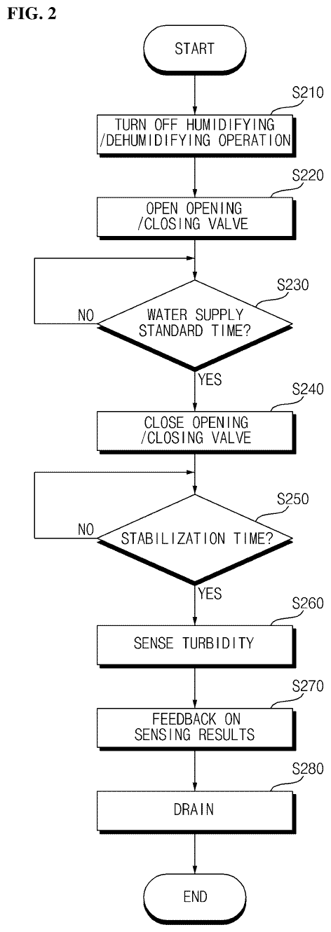

FIG. 2 is a flowchart of an operating method of an air conditioner according to an embodiment of the present disclosure.

Referring to FIG. 2, the opening/closing valve 21 operates to open the sensor water-supply flow path 27a connecting the water tank 10 and the turbidity sensor 25 arranged under the lower end portion of the water tank 10 (S220). The opening/closing valve 21 may open or close the sensor water-supply flow path 27a according to the control of the controller 30.

In order to improve sensing accuracy, it is more preferable that the controller 30 turn off the humidifying or dehumidifying operation of the air conditioner 1 (S210) before opening the sensor water-supply flow path 27a (S220).

When a preset water supply standard time has elapsed (S230), the opening/closing valve 21 operates to close the sensor water-supply flow path 27a (S240). Here, the water supply standard time may be decided in advance based on the amount of water needed for sensing by the turbidity sensor 25 and the speed at which water enters an interior of the turbidity sensor 25. At the stage where the valve operates to close the sensor water-supply flow path (S240), when a preset water supply standard time has elapsed after the sensor water-supply flow path 27a is opened (S220) or a certain amount of water is filled in the internal chamber of the turbidity sensor 25, the operating state of the opening/closing valve 21 may be changed.

Thereafter, when a sufficient stabilization time has elapsed for bubbles and vortices that may be generated by the internal inflow of water to disappear (S250), the turbidity sensor 25 may sense the turbidity of water supplied from the water tank 10 (S260). According to an embodiment, the turbidity sensor 25 may directly determine the contamination level of water based on the turbidity sensing value. Alternatively, the controller 30 may receive the turbidity sensing value of the turbidity sensor 25 and discriminate the contamination level of water.

The controller 40 may be controlled to perform a feedback operation based on the sensing value of the turbidity sensor 25 (S270).

When the discriminated contamination level is included in the first section, the controller 40 may display the discriminated contamination level and control the UV module 60 to irradiate ultraviolet rays to an interior of the water tank 10.

When the discriminated contamination level is included in the second section that is higher than the first section, the controller 40 may control the output module 40 that displays the discriminated contamination level information and information guiding the washing of the water tank 10. In addition, the communication module 50 may transmit a signal to a server to transmit the washing guide image of the water tank 10 to a preset device according to the control of the controller 40. The server transmits the washing guide image to a preset device (for example, a user terminal such as a smartphone, or the air conditioner 1), and a user may conveniently learn about situations requiring washing and a method for washing the water tank 10 by viewing the received washing guide image.

When the discriminated contamination level is included in the third section that is higher than the second section, the controller 40 may control the output module 40 to display the discriminated contamination level information and control the communication module 50 to automatically request a care service in response to the discriminated contamination level.

After sensing (S260), the turbidity sensor 25 may discharge water (S280). The drainage of the turbidity sensor 25 (S280) may be performed separately from the feedback operation (S270). Accordingly, the feedback operation (S270) and the drainage (S280) may be performed one after the other or may be performed simultaneously.

The turbidity sensor 25 and the water tank 10 may be formed as an integrated or detachable type.

The integrated type is a case where the turbidity sensor 25 is connected to the lower end portion of the water tank 10, and an inlet of the sensor water-supply flow path 27a or an outlet structure connected to the inlet of the sensor water-supply flow path 27a may be formed at the lower end portion of the water tank 10. In addition, the opening/closing valve 21 is arranged in the sensor water-supply flow path 27a. More preferably, the opening/closing valve 21 may be arranged at the inlet of the sensor water-supply flow path 27a.

The detachable type is a case where the turbidity sensor 25 is connected to some parts of the main body of the air conditioner 1 other than the detachable water tank 10, and a functional unit fastened to the opening/closing valve 21 and the pump 23 may be formed at the lower end portion of the water tank 10. After the water tank 10 is connected to the main body of the air conditioner 1, the functional units of the opening/closing valve 21 and the pump 23 may also be linked and operated.

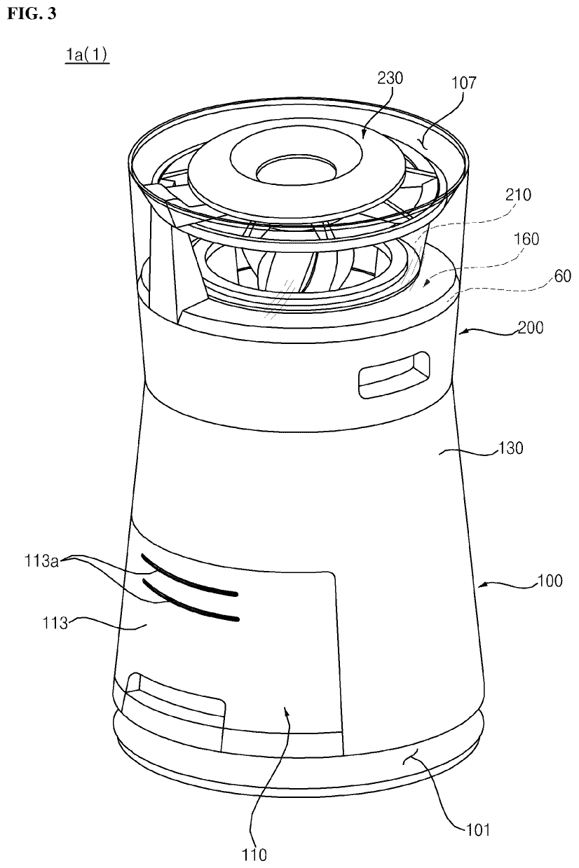

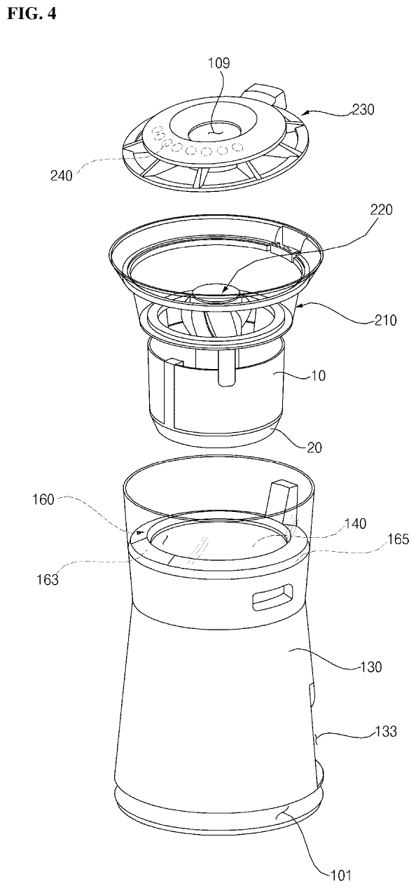

FIG. 3 is a perspective view of an air conditioner according to an embodiment of the present disclosure. FIG. 4 is an exploded perspective view of the air conditioner of FIG. 3. FIGS. 3 and 4 illustrate a case where the water tank sensing module 20 including the turbidity sensor 25 is applied to an air conditioner la provided with a humidifying function and an air purifying function.

Referring to FIGS. 3 and 4, the water tank sensing module 20 is integrally arranged at a lower end of the detachable water tank 10. Accordingly, when the water tank 10 is detached from the main body of the air conditioner la body (for example, a mounting body 140), the water tank sensing module 20 may also be detached from the main body.

Alternatively, the water tank sensing module 20 may be formed as a detachable type. In this connection, even when the water tank 10 is detached from the main body of the air conditioner 1a, the water tank sensing module 20 may be fixed to the main body of the air conditioner la (for example, the mounting body 140).

Referring to FIGS. 3 and 4, the air conditioner 1a includes a purification module 100 that sucks in external air and then purifies the same, and a humidification module 200 that provides moisture to the air purified in the purification module 100 to humidify the same.

The purification module 100 includes: a base body 130 that supports the air conditioner from the floor and forms an exterior; a filter assembly 110 that is detachably installed in the base body 130 and purifies the air; and the blower module 70 that is arranged inside the base body 130 and causes the air to flow.

The base body 130 forms the exterior of the air conditioner. The upper portion of the base body 130 is formed of a transparent material that allows a user to see inside. The base body 130 has an intake port 101 formed at a lower portion through which external air is sucked in. The base body 130 has a filter installation opening unit 133 formed on one side into which the filter assembly 110 is inserted. The base body 130 includes the cylindrical mounting body 140 arranged on an inner upper portion into which the water tank 10 is inserted. A display module 160 is arranged on an upper side of the mounting body 140. A space is formed between the mounting body 140 and the display module 160, into which air blown by the blower module 70 enters.

The blower module 70 causes air to flow so that air is sucked in through the intake port 101 and discharged through a discharge port 107. The blower module 70 includes a blower fan (not shown) that blows air and a blower motor (not shown) that rotates the blower fan. The air blown from the blower fan flows to the humidification module 200. The blower motor may rotate the blower fan at various rotation speeds.

The filter assembly 110 purifies the air sucked in through the intake port 101. The filter assembly 110 is arranged at a lower portion of a blower unit. The purified air from the filter assembly 110 flows to the blower module 70.

The filter assembly 110 includes a filter cover 113 that shields the filter installation opening unit 133 when installed in the base body 130, and an electric dust collector module 15 that electrifies foreign substances (dust) in the air to purify the air. The filter assembly 110 may further include a prefilter (not shown) that filters foreign substances in the air sucked in through the intake port 101 and a deodorizing filter (not shown) that deodorizes odors in the air. The filter cover 113 may have an open sensing opening unit 113a formed to allow external air to enter through an air condition sensor (not shown), which will be described later.

The humidification module 200 includes: a visual body 210 that is detachably combined with the purification module 100 and formed of a transparent material that allows a user to see inside; the cylindrical water tank 10 that is combined with the visual body 210 and stores water; a water turbine unit 220 that sucks in water inside the water tank 10 and sprays the same; a humidifying medium (not shown) that wets with the water sprayed from the water turbine unit 220 and contains moisture and humidifies the air passing therethrough; and a top cover assembly 230 that is arranged on a upper end of the visual body 210. The humidifying medium contains moisture and humidifies the air passing therethrough.

The visual body 210 is arranged in an upper portion of the base body 130. The visual body 210 is combined at its lower end with the water tank 10. The visual body 210 reflects and scatters water sprayed from the water turbine unit 220.

The water tank 10 stores water. The water tank 10 is inserted into the mounting body 140 of the base body 130. The upper portion of the water tank 10 may be combined with the visual body 210 and may be detached from the purification module 100 together with the visual body 210. A space is formed between the visual body 210 and the water tank 10, the blower fan 24 of the blower module 70 blows air, and air passing between the mounting body 140 and the display module 160 enters.

The top cover assembly 230 covers an upper side of the visual body 210. The top cover assembly 230 is detachably combined with the visual body 210. The discharge port 107 is formed in the top cover assembly 230 through which humidified air from the water turbine unit 220 is discharged to the outside. A water supply hole (not shown) may be formed in the center of the top cover assembly 230 to supply the fallen water to the water tank 10. A user may supply water to the water tank 10 by pouring water into the center of the top cover assembly 230.

A manipulation module 240 receives commands from a user. The manipulation module 240 is arranged on an upper surface of the top cover assembly 230. Through the manipulation module 240, the user inputs various commands to control the driving of the air conditioner.

The display module 160 displays the state of the air conditioner. The display module 160 is formed in a ring shape and arranged inside the base body 130. The display module 160 is arranged so that a user may see inside through a portion formed of a transparent material on an upper portion of the base body 130. The user may check the air condition or the current driving mode and other settings through the display module 160. The mounting body 140 is arranged on a lower side of the display module 160.

The display module 160 includes a display unit 163 that displays the state of the air conditioner using icons or letters, and a lighting unit 165 that displays the state of the air conditioner using lights of various colors. The display unit 163 displays the air condition, driving mode, or other settings using icons or letters. The lighting unit 165 irradiates light of various colors to display the air condition.

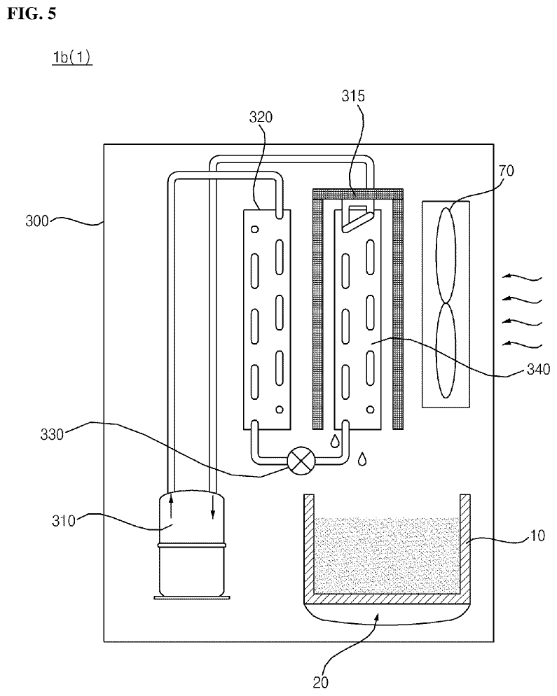

FIG. 5 is a conceptual diagram of an air conditioner according to another embodiment of the present disclosure. FIG. 5 illustrates a case where the water tank sensing module 20 including the turbidity sensor 25 is applied to a dehumidifier 1b provided with a dehumidifying function.

Referring to FIG. 5, the dehumidifier 1b may include a case 350 forming an exterior. The case 350 may accommodate components of the dehumidifier 1b inside.

The case 350 may include an air inlet (not shown) to allow air to enter an interior of the dehumidifier 1b. The case 350 may include an air outlet (not shown) that allows air to be discharged outside the dehumidifier 1b.

The case 350 is open on one side so that the water tank 10, which is a condensate storage container, may be arranged detachably from the dehumidifier 1b. The water tank sensing module 20 may be formed as a detachable or integrated type at a lower side of the lower end portion of the water tank 10.

The water tank 10 may be formed so that the surface facing an evaporator 340 is entirely or partially open. The water tank 10 may be formed with an open upper side. The water tank 10 may include a funnel shape on an upper side to effectively collect the condensate generated and falling from the evaporator 340.

The water tank 10 may be located at a lower side of the evaporator 340 in the direction of gravity, and may collect and store condensate generated and falling from the evaporator 340. The water tank 10 may be provided in a detachable manner.

The water tank 10 may be provided so as to be openable on one side of the dehumidifier 1b. The water tank 10 may be provided so as to be openable in a sliding and/or rotating manner.

A compressor 310 receives power from a power supply source (such as an engine or motor) and drives to suck in and compress the low-temperature, low-pressure gaseous refrigerant discharged from the evaporator 340 and discharge the same to a condenser 320 as a high-temperature, high-pressure gaseous state.

Next, the condenser 320 condenses the refrigerant, which is in a high-temperature, high-pressure gaseous state discharged from the compressor 310, into a medium-temperature, high-pressure liquid by exchanging heat with the outside air and discharges the same through an expansion valve 330.

The expansion valve 330 rapidly expands the refrigerant, which is in a medium-temperature, high-pressure liquid state that has passed through a heat exchanger 315, through a throttle action and sends the same to the evaporator 340 as a low-temperature, low-pressure liquid state. In this connection, there is no change in enthalpy.

The evaporator 340 evaporates the refrigerant, which is in a low-temperature, low-pressure liquid state that has been throttled by the expansion valve 330, by exchanging heat with the outside air entering the dehumidifier 1b, thereby cooling the air through the heat absorption action due to the latent heat of vaporization of the refrigerant. According to an embodiment, the dehumidifier 1b may include the heat exchanger 315 that absorbs heat from air entering the evaporator 340 and provides the same to air discharged from the evaporator 340, thereby improving dehumidification efficiency.

The cooling air reaches the dew point, condenses in the evaporator 340, falls by gravity, and is stored in the water tank 10.

In addition, the refrigerant that evaporates in the evaporator 340 and becomes a low-temperature, low-pressure gaseous state is sucked back into the compressor 310 and recirculates the refrigeration cycle as described above.

In addition, in the refrigerant circulation process as described above, air that has passed through the evaporator 340 arranged upstream in the air flow direction and has its temperature lowered passes through the condenser 320 arranged downstream and has its temperature raised again through heat exchange with the refrigerant and is discharged outside the dehumidifier 1b.

The dehumidifier 1b may include the blower fan 70. The dehumidifier 1b may include one or more blower fans 70. The dehumidifier 1b may include the blower fan 70 that inhales air through the air inlet and discharges the same through the air outlet.

The blower fan 70 may ensure smooth air inflow into the dehumidifier 1b. The blower fan 70 may smoothly discharge dehumidified air out of the dehumidifier.

The blower fan 70 may blow air into an air flow path inside the dehumidifier 1b. The air flowing by the blower fan 70 may be sequentially heat-exchanged with the evaporator 340 and the condenser 320 while passing through the air flow path inside the dehumidifier 1b and then discharged indoors through the air outlet of the dehumidifier 1b.

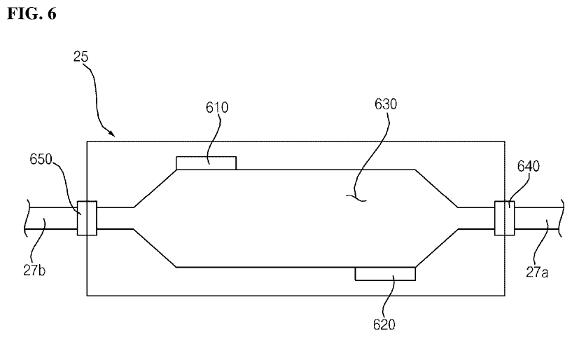

FIG. 6 is a conceptual diagram of a turbidity sensor according to an embodiment of the present disclosure.

The turbidity sensor 25 according to an embodiment of the present disclosure senses turbidity (contamination level) through optical sensing.

Optical sensing methods for measuring turbidity include a transmitted light method and a scattered light method. The transmitted light method is configured to sense turbidity by irradiating light to a fluid, receiving the light transmitted through the fluid, and processing data. The scattered light method is a method of sensing turbidity by receiving scattered light and converting the same into data, and is subdivided according to a method of generating scattered light and a method of processing data on received light.

Referring to FIG. 6, the turbidity sensor 25 may include a light source unit 610 including a light source (for example, a laser light source) and a light receiving unit 620 that receives scattered light.

In addition, the turbidity sensor 25 may include a chamber 630 filled with water supplied from the water tank 10. The chamber 630 may be connected to the sensor water-supply flow path 27a through an inlet 640 and to the sensor drainage flow path 27b through an outlet 650. The light source unit 610 may irradiate light to an interior of the chamber 630, and the light receiving unit 620 may receive scattered light scattered in the chamber 630.

The turbidity sensor 25 may sense scattered light emitted from a visible light laser source that is reflected and dispersed by floating substances in water and output the same as a signal value. Scattered light increases proportionally with the amount of particles in a fluid, but may vary depending on the behavior of the particles, the fluid condition (physical environment such as the generation of bubbles or vortices), or external environment such as vibration. Accordingly, the turbidity sensor 25 may proceed with a sensing process after water enters an interior of the chamber 630 and a stabilization time has elapsed.

The light source unit 610 may irradiate the light when the chamber 630 is filled with the entered water to a certain water level or higher. More preferably, the chamber 630 is filled with the entered water to a certain water level or higher, and after the stabilization time has elapsed, the light source unit 610 may irradiate light and perform a sensing process.

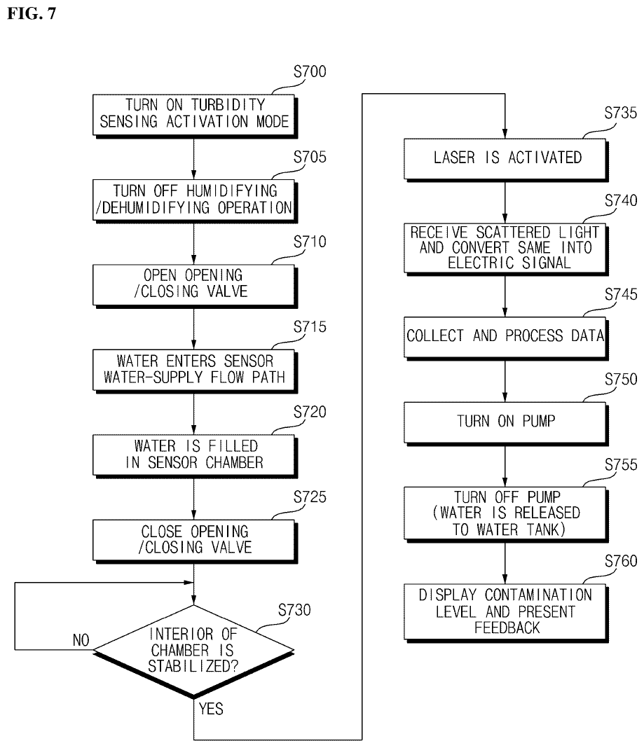

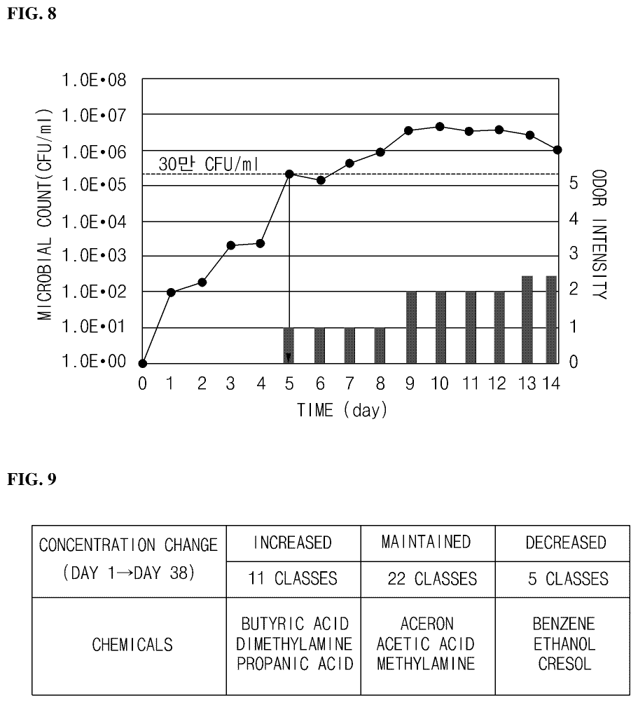

FIG. 7 is a flowchart of an operating method of an air conditioner according to an embodiment of the present disclosure. FIGS. 8 to 11 are diagrams for reference in describing the operation of an air conditioner according to an embodiment of the present disclosure.

Referring to FIG. 7, when the turbidity sensing activation mode is turned on (S700), the controller 30 may turn off the humidification or dehumidification operation of the air conditioner 1 (S705).

Thereafter, under the control of the controller 30, the opening/closing valve 21 operates to open the sensor water-supply flow path 27a connecting the water tank 10 and the turbidity sensor 25 (S710).

When the sensor water-supply flow path 27a is opened (S710), water enters the sensor water-supply flow path 27a from the water tank 10 (S715), and water is filled in the chamber 630 inside the turbidity sensor 25 (S720). The water enters the chamber 630 at a constant flow rate per second/minute.

When a preset water supply standard time has elapsed, the opening/closing valve 21 operates to close the sensor water-supply flow path 27a (S7250). Herein, the water supply standard time may be decided in advance based on the amount of water needed for sensing by the turbidity sensor 25 and the speed at which water enters an interior of the turbidity sensor 25.

Thereafter, when a sufficient stabilization time has elapsed for bubbles and vortices that may be generated by the internal inflow of water to disappear (S730), the turbidity sensor 25 may sense turbidity (contamination level).

The sensing may include: irradiating light to an interior of the chamber 630 (S735); receiving scattered light scattered in the chamber 630 (S740); and analyzing a pattern of the received scattered light (S745). Pattern analysis of scattered light may be performed by an artificial neural network trained using deep learning techniques. An artificial neural network may be trained to analyze the pattern of scattered light and output the turbidity (contamination level) of water. The pre-trained artificial neural network may be mounted on the turbidity sensor 25 or on a controller 25 of the air conditioner 1.

When the stabilization time has elapsed (S730), the laser of the light source unit 610 is activated and light is irradiated to an interior of the chamber 630 (S735). The light receiving unit 620 receives scattered light and converts the same into an electric signal (S740), and a microcomputer (not shown) of the turbidity sensor 25 collects/processes data to sense turbidity (S745).

According to an embodiment, the turbidity sensor 25 may directly discriminate the contamination level of water based on a sensing value. Alternatively, the controller 30 may receive the sensing value of the turbidity sensor 25 and discriminate the contamination level of water.

The pump 23 for drainage is turned on to start draining the water discharged from the chamber 630 to the water tank 10 (S750). When the releases of water to the water tank 10 is completed, the pump 23 may be turned off (S755). The controller 40 may be controlled to perform a feedback operation including displaying a contamination level based on the sensing value of the turbidity sensor 25 (S760).

The odor in the water tank is caused by the propagation of microorganisms and biofilms, so ultraviolet sterilization and reduction of nutrient inflow are necessary to suppress the same.

FIG. 8 shows the results of testing the odor intensity and microbial concentration that occur when a product is driven under adverse conditions. Referring to FIG. 8, the odor intensity increases in proportion to the microbial concentration, and the odor occurs at 300,000 CFU/ml or more. FIG. 9 illustrates changes in the concentration of chemicals according to product activation. It may be inferred that chemicals produced by microorganisms primarily increase and that the cause of the odor in the water tank 10 is related to the metabolism of microorganisms.

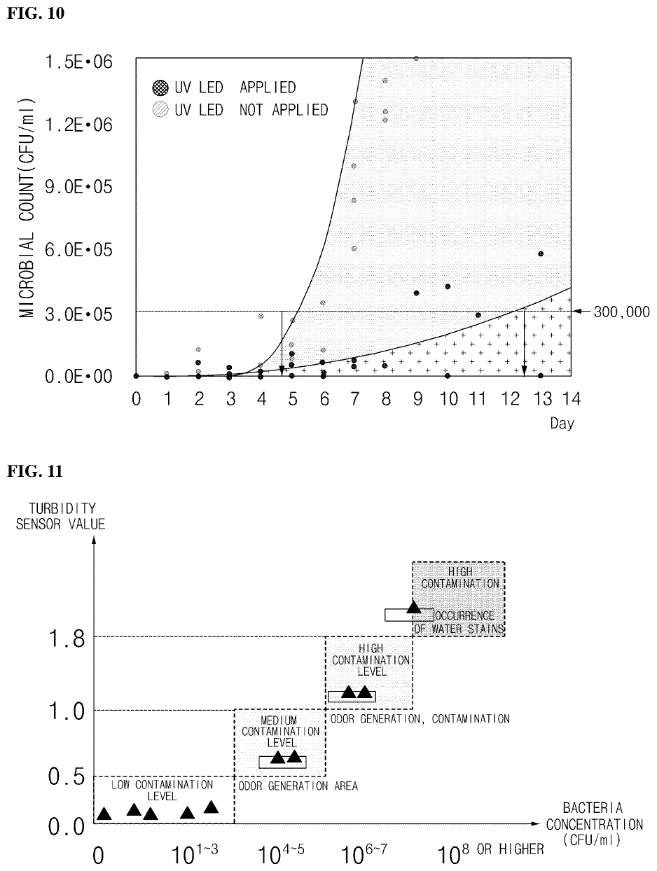

FIG. 10 illustrates the results of an experiment verifying the effect of floating microorganisms on ultraviolet irradiation. FIG. 10 illustrates the number of microorganisms by date when an LED that irradiates ultraviolet rays was applied and when not applied. Referring to FIG. 8, it is expected that the propagation of microorganisms will be suppressed by the sterilizing effect of ultraviolet rays, thereby preventing/delaying the occurrence of water odor.

FIG. 11 illustrates the relationship between turbidity sensor values and contamination levels, and exemplifies the level discrimination of microorganisms in the water tank and the contamination level discrimination section according to a turbidity sensor output value.

When the contamination level is ‘low’ or lower, automatic washing/sterilization logic may be performed using the UV module 60. Accordingly, it is possible to prevent the contamination level of the water tank 10 from increasing in advance.

When the contamination level is ‘medium’, user washing progress information (such as linking to a care service or sending washing video) may be displayed as a section where odor may occur.

In addition, when the contamination level is ‘high’ or higher, the water may have already been contaminated to the point of odor and water stains may have occurred, so the care service may be automatically called immediately and an alarm may be provided to a user.

According to at least one of the embodiments of the present disclosure, the contamination level of water in the water tank may be accurately discriminated and management feedback may be presented, thereby improving internal hygiene performance and usability.

Hereinbefore, although preferred embodiments of the present disclosure have been illustrated and described, the present disclosure is not limited to the specific embodiments described above, and it goes without saying that persons having ordinary skills in the technical field to which the present disclosure pertains may implement the present disclosure by various modifications thereof without departing from gist of the present disclosure defined by the claims, and such modifications are not to be construed individually from the technical spirit and scope of the present disclosure.

1. A air conditioner, comprising:
a water tank in which water that is generated or required in the process of conditioning the air is accommodated;
a turbidity sensor arranged under the lower end portion of the water tank;
a sensor water-supply flow path through which the water accommodated in the water tank flows to the turbidity sensor;
a valve opening/closing the sensor water-supply flow path;
a sensor drainage flow path through which the water discharged from the turbidity sensor flows; and
a pump allowing the water discharged from the turbidity sensor to flow to the water tank through the sensor drainage flow path.

2. The air conditioner of claim 1, wherein the turbidity sensor comprises:
a chamber filled with water supplied from the water tank;
a light source unit comprising a light source that irradiates light to an interior of the chamber; and
a light receiving unit that receives scattered light scattered in the chamber.

3. The air conditioner of claim 2, wherein the light source unit irradiates the light when the chamber is filled to a certain water level or higher.

4. The air conditioner of claim 1, wherein the turbidity sensor senses and output turbidity of water supplied from the water tank after a preset stabilization time has elapsed.

5. The air conditioner of claim 1, wherein the valve opens the sensor water-supply flow path and closes the sensor water-supply flow path after a preset water supply standard time has elapsed.

6. The air conditioner of claim 1, further comprising a controller that discriminates a microbial contamination level in the water tank based on a sensing value of the turbidity sensor and controls to perform a feedback operation in response to the discriminated microbial contamination level.

7. The air conditioner of claim 6, wherein the controller turns off an operation of the air conditioner and controls an opening of the valve.

8. The air conditioner of claim 6, further comprising a UV module that irradiates ultraviolet rays to an interior of the water tank when the discriminated contamination level is included in a first section.

9. The air conditioner of claim 8, further comprising an output module that displays washing-related information corresponding to the discriminated contamination level when the discriminated contamination level is included in a second section higher than the first section.

10. The air conditioner of claim 9, further comprising a communication module that automatically requests a care service in response to the discriminated contamination level when the discriminated contamination level is included in a third section higher than the second section.

11. A method for operating an air conditioner, the method comprising:
operating a valve to open a sensor water-supply flow path connecting a water tank in which water that is generated or required in the process of conditioning the air is accommodated and a turbidity sensor arranged under the lower end portion of the water tank;
operating the valve to close the sensor water-supply flow path;
using the turbidity sensor to sense turbidity of water supplied from the water tank when a stabilization time has elapsed; and
performing a feedback operation based on a sensing value of the turbidity sensor.

12. The method of claim 11, further comprising draining of discharging water from the turbidity sensor.

13. The method of claim 11, further comprising turning off an operation of the air conditioner before opening the sensor water-supply flow path.

14. The method of claim 11, wherein the operation of the valve to close the sensor water-supply flow path is configured to change an operating state of the valve when a preset water supply standard time has elapsed after opening or when an internal chamber of the turbidity sensor is filled with water exceeding a certain amount.

15. The method of claim 11, wherein the use of the turbidity sensor to sense the turbidity of the water supplied from the water tank comprises:
irradiating light to an interior of the chamber;
receiving scattered light scattered in the chamber; and
analyzing a pattern of the received scattered light.

16. The method of claim 11, wherein the performance of the feedback operation comprises displaying a discriminated contamination level and irradiating ultraviolet rays to an interior of the water tank when the discriminated contamination level is included in a first section.

17. The method of claim 16, wherein the performance of the feedback operation comprises displaying discriminated contamination level information and information guiding washing of the water tank when the discriminated contamination level is included in a second section higher than the first section.

18. The method of claim 17, wherein the performance of the feedback operation comprises transmitting a signal to a server to transmit a washing guide image of the water tank to a preset device.

19. The method of claim 16, wherein the performance of the feedback operation comprises displaying discriminated contamination level information and automatically requesting a care service in response to the discriminated contamination level when the discriminated contamination level is included in a third section higher than the second section.
